(19) United States
(12) Patent Application Publication
(10) Pub. No.: US 2002/0193865 A1
(43) Pub. Date: Dec. 19, 2002

(54) Radiopaque stent

(76) Inventors: Herbert Radisch, San Diego, CA (US); Thomas Trozera, Del Mar, CA (US); Charles Horace Craig, Lakeside, CA (US)

Correspondence Address: MICHAEL E. KLICPERA, PO BOX 573, LA JOLLA, CA, 92038-0573, US

(21) Appl. No.: 09/823,308

(22) Filed: Mar. 30, 2001

(51) Int. Cl. A61F 2/06

(52) U.S. Cl. 623/1.15; 623/1.34

(57) ABSTRACT

The present invention is directed towards a stent fabricated from an austenitic 300 series stainless steel alloy having improved radiopaque characteristics. The modified stainless steel alloy consists essentially of, in weight percent, about

| C | Mn | Si | P | S |
|---|---|---|---|---|
| ≦0.030 | ≦2.000 | ≦0.750 | ≦0.023 | ≦0.010 |
| Cr | Mo | Ni | Fe | “X” |
| 12.000- | 000- | 10.000- | 46.185- | 2.000- |
| 20.000 | 3 000 | 18.000 | 74.000 | 10.000 |

whereby variable “X” could be comprised from a group consisting of Gold, Osmium, Palladium, Platinum, Rhenium, Tantalum, or Tungsten. The alloy provides a unique combination of strength, ductility, corrosion resistance, and other mechanical properties which also has improved radiopaque characteristics in the thin sections necessary to manufacture a stent and is low toxicity.

### BACKGROUND OF THE INVENTION

Cardiovascular disease is commonly accepted as being one of the most serious health risks facing our society today. Diseased and obstructed coronary arteries can restrict the flow of blood and cause tissue ischemia and necrosis. While the exact etiology of sclerotic cardiovascular disease is still in question, the treatment of narrowed coronary arteries is more defined. Surgical construction of coronary artery bypass grafts (CABG) is often the method of choice when there are several diseased segments in one or multiple arteries. Conventional open heart surgery is, of course, very invasive and traumatic for patients undergoing such treatment. In many cases, less traumatic, alternative methods are available for treating cardiovascular disease percutaneously. These alternate treatment methods generally employ various types of balloons (angioplasty) or excising devices (atherectomy) to remodel or debulk diseased vessel segments. A further alternative treatment method involves percutaneous, intraluminal installation of one or more expandable, tubular stents or prostheses in sclerotic lesions. Intraluminal endovascular prosthetic grafting is an alternative to conventional vascular surgery. Intraluminal endovascular grafting involves the percutaneous insertion into a blood vessel of a tubular prosthetic graft and its delivery via a catheter to the desired location within the vascular system. The alternative approach to percutaneous revascularization is the surgical placement of vein, artery, or other by-pass segments from the aorta onto the coronary artery, requiring open heart surgery, and significant morbidity and mortality. Advantages of the percutaneous revascularization method over conventional vascular surgery include obviating the need for surgically exposing, removing, replacing, or by-passing the defective blood vessel, including heart-lung by-pass, opening the chest, and general anesthesia.

Stents or prostheses are known in the art as implants which function to maintain patency of a body lumen in humans and especially to such implants for use in blood vessels. They are typically formed from a cylindrical metal mesh which expand when internal pressure is applied. Alternatively, they can be formed of wire wrapped into a cylindrical shape. The present invention relates to an improved stent design which by its specifically configured struts can facilitate the deployment and embedment of the stent within a vessel and is constructed from a manufacturing process which provides a controlled and superior stress yield point and ultimate tensile characteristics.

Stents or prostheses can be used in a variety of tubular structures in the body including, but not limited to, arteries and veins, ureters, common bile ducts, and the like. Stents are used to expand a vascular lumen or to maintain its patency after angioplasty or atherectomy procedures, overlie an aortic dissecting aneurysm, tack dissections to the vessel wall, eliminate the risk of occlusion caused by flaps resulting from the intimal tears associated with primary interventional procedure, or prevent elastic recoil of the vessel.

Stents may be utilized after atherectomy, which excises plaque, cutting balloon angioplasty, which scores the arterial wall prior to dilatation, or standard balloon angioplasty to maintain acute and long-term patency of the vessel.

Stents may be utilized in by-pass grafts as well, to maintain vessel patency. Stents can also be used to reinforce collapsing structures in the respiratory, biliary, urological, and other tracts.

Further details of prior art stents can be found in U.S. Pat. No. 3,868,956 (Alfidi et. al.); U.S. Pat. No. 4,739,762 (Palmaz); U.S. Pat. No. 4,512,338 (Balko et. al.); U.S. Pat. No. 4,553,545 (Maass et. al.); U.S. Pat. No. 4,733,665 (Palmaz); U.S. Pat. No. 4,762,128 (Rosenbluth); U.S. Pat. No. 4,800,882 (Gianturco); U.S. Pat. No. 4,856,516 (Hillstead); U.S. Pat. No. 4,886,062 (Wiktor); U.S. Pat. No. 5,102,417 (Palmaz); U.S. Pat. No. 5,104,404 (Wolff); U.S. Pat. No. 5,192,307 (Wall); U.S. Pat. No. 5,195,984 (Schatz); U.S. Pat. No. 5,282,823 (Schwartz et. al.); U.S. Pat. No. 5,354,308 (Simon et. al.); U.S. Pat. No. 5,395,390 (Simon et. al), U.S. Pat. No. 5,421,955 (Lau et. al.); U.S. Pat. No. 5,443,496 (Schwartz et. al.); U.S. Pat. No. 5,449,373 (Pinchasik et. al.); U.S. Pat. No. 5,102,417 (Palmaz); U.S. Pat. No. 5,514,154 (Lau et. al); and U.S. Pat. No. 5,591,226 (Trerotola et. al.).

In general, it is an object of the present invention to provide a stent or prosthesis which can be readily delivered to, expanded and embedded into an obstruction or vessel wall with radiopaque characteristics for fluoroscopic observations during all phases of the interventional procedure.

It is another object of the present invention to provide a stent that is fabricated from austenitic 300 series stainless steel alloy that provides better radiopacity than is provided by the known austenitic stainless steels.

### SUMMARY OF THE INVENTION

The present invention is directed to a radiopaque stent which is relatively flexible along its longitudinal axis to facilitate delivery through tortuous body lumens, but which is stiff and stable enough radially in an expanded condition to maintain the patency of a body lumen such as an artery when implanted therein.

The invention generally relates to virtually any stent design that is manufactured from stainless steel or other materials not having inherent radiopacity properties but which requires increased radiopacity characteristics. For the purposes of this disclosure, the terms radiopacity or radiopaque refer to a characteristic or material that is opaque to X-ray radiation that renders the material visible under fluoroscopy. Stents are generally delivered and deployed using standard angioplasty techniques (such as employing an over-the-wire or rapid exchange delivery balloon) within the coronary vasculature of the human subject. In this clinical setting, the interventionalist uses angiographic and fluoroscopic techniques that employ X-rays and materials that are radiopaque to the X-rays to visualize the location or placement of the particular device with the human vasculature. Typically stents are fabricated from a variety of stainless steels, with the 316 series representing a large percentage of the stainless steel used to fabricate currently marketed stents. The typical composition of 316 series stainless steel is shown in Table I.

TABLE I

| | Component (%) | | | | | | | | |
|---|---|---|---|---|---|---|---|---|---|
| | C | Mn | Si | P | S | Cr | Mo | Ni | Fe |
| Standard 316 | 0.020 | 1.760 | 0.470 | 0.014 | 0.002 | 17.490 | 2.790 | 14.680 | 62.774 |

While the austenitic 300 series stainless steel used for fabricating stents has several beneficial characteristics, such as strength, flexibility, fatigue resistance, biocompatibility, etc. rendering it a good material to make an intravascular stent, one significant disadvantage of 316 series stainless steel, as well as other 300 series of stainless steel, is that they have relatively low radiopaque qualities and therefore not readably visual under fluoroscopic observation. It is desirable to utilize a material such a 300 series stainless steel because of its physical characteristics in fabricating a stent. Yet the struts of the stent must be relatively thin and therefore are poorly visualized under the X-ray fluoroscope. A need has arisen to modify the stainless steel composition so it has radiopaque properties while at the same time maintaining those characteristics which render it as a material of choice for fabricating stents.

In order to increase the radiopaque characteristics of series 300 stainless steel in the thin sections required to fabricate an intravascular stent, an alloy containing varying amounts of elements that have dense mass and radiopaque characteristics will be incorporated into the series 300 chemical structure. The chemical make-up of standard series 300 stainless steel, using series 316 as an example, along with the possible chemical ranges of various such alloys are shown on the following Table.

TABLE II

| | Component (%) | | | | | | | | | |
|---|---|---|---|---|---|---|---|---|---|---|
| | C | Mn | Si | P | S | Cr | Mo | Ni | Fe | x |
| Standard 316 | 0.020 | 1.760 | 0.470 | 0.014 | 0.002 | 17.490 | 2.790 | 14.680 | 62.774 | 0.000 |
| Modified 316 | ≦0.030 | ≦2.000 | ≦0.750 | ≦0.023 | ≦0.010 | 12.000- | 000- | 10.000- | 46.185- | 2.000- |
| | | | | | | 20.000 | 3.000 | 18.000 | 74.000 | 10.000 |

Variable “X” could be comprised of or a combination of Au, Os, Pd, Pt, Re, Ta or W.

The stent, embodying features of the present invention, can be readily delivered to the desired lumenal location by mounting it on an expandable member of a delivery catheter, for example, a balloon or mechanical dilatation device, and passing the catheter/stent assembly through the body lumen to the site of deployment.

Other features and advantages of the present invention will become more apparent from the following detailed description of the invention, when taken in conjunction with the accompanying exemplary drawings.

### BRIEF DESCRIPTION OF THE DRAWINGS

FIG. 1 is a schematic view of the present invention in its intended operational environment.

FIGS. 2-8 are illustrations of various designs of prior art surgical stents that could be used in conjunction with radiopaque stainless steel alloy.

FIG. 9 is a schematic view of the present invention showing the radiopaque stent and delivery catheter in a proximal position relative to the lesion. Also shown is the corresponding image of the proximally placed radiopaque stent on a typical cine angiogram or fluoroscopic equipment.

FIG. 10 is a schematic view of the present invention showing the radiopaque stent and delivery catheter centered within the lesion in a contracted configuration. Also shown is the corresponding image of the contracted radiopaque stent centered within the lesion on a typical cine angiogram or fluoroscopic equipment.

FIG. 11 is a schematic view of the present invention showing the radiopaque stent and delivery catheter centered within the lesion in an expanded configuration. Also shown is the corresponding image of the expanded radiopaque stent centered within the lesion on a typical cine angiogram or fluoroscopic equipment.

FIG. 12 is a schematic view of the present invention showing the expanded and deployed radiopaque stent embedded within the lesion. Also shown is the corresponding image of the embedded radiopaque stent deployed within the lesion on a typical cine angiogram or fluoroscopic equipment.

### DETAILED DESCRIPTION

The stent according to the present invention is fabricated from an austenitic stainless steel series 300 alloy compound which replaces a portion of the iron or molybdenum component of the 300 series with one or combination of several elements containing radiopaque properties. Examples of such elements are gold (Au), osmium (Os), palladium (Pd), platinum (Pt), rhenium (Re), tantalum (Ta) or tungsten (W). This group consists of elements with dense masses. The dense mass provides these materials with improved absorption of X-rays thus providing improved radiopaque characteristics. By including one or more of these elements in a series 300 stainless steel, thereby creating an unique alloy for the present invention, X-rays employed in angiogram procedures or cineograms allow the visualization of the stent during all phases of a standard clinical procedure. The alloy for fabricating stents contains a range of 2.0 to 10.0 percent of one or more of these radiopaque elements, with a preferred range of 4.0 to 5.0 percent. Replacing too much of the radiopaque element with the iron or molybdenum component could possibly decrease the beneficial qualities of 300 series stainless steel for manufacturing stents without contributing significantly improved radiopaque characteristics. It is anticipated that various combinations of the radiopaque elements can be used to replace the iron or molybdenum component without adversely affecting the ability to form austenite.

The foregoing, as well as additional objects and advantages of the present invention, are achieved by employing an unique austenitic stainless steel alloy. The formulations of these alloys are compared with standard 316 stainless steel and summarized in Tables III through X below, containing in weight percent, about:

TABLE III

| | Component (%) | | | | | | | | | |
|---|---|---|---|---|---|---|---|---|---|---|
| | C | Mn | Si | P | S | Cr | Mo | Ni | Fe | x |
| Standard 316 | 0.020 | 1.760 | 0.470 | 0.014 | 0.002 | 17.490 | 2.790 | 14.680 | 62.774 | 0.000 |
| Modified 316 | ≦0.030 | ≦2.000 | ≦0.750 | ≦0.023 | ≦0.010 | 12.000- | 000- | 10.000- | 46.185- | 2.000- |
| | | | | | | 20.000 | 3.000 | 18.000 | 74.000 | 10.000 |

Where variable “X” could be comprised of or a combination of Au, Os, Pd, Pt, Re, Ta or W.

TABLE IV

| | Component (%) | | | | | | | | | |
|---|---|---|---|---|---|---|---|---|---|---|
| | C | Mn | Si | P | S | Cr | Mo | Ni | Fe | Au |
| Standard 316 | 0.020 | 1.760 | 0.470 | 0.014 | 0.002 | 17.490 | 2.790 | 14.680 | 62.774 | 0.000 |
| Modified 316 | ≦0.030 | ≦2.000 | ≦0.750 | ≦0.023 | ≦0.010 | 12.000- | .000- | 10.000- | 46.185- | 2.000- |
| | | | | | | 20.000 | 3.000 | 18.000 | 74.000 | 10.000 |

TABLE V

| | Component (%) | | | | | | | | | |
|---|---|---|---|---|---|---|---|---|---|---|
| | C | Mn | Si | P | S | Cr | Mo | Ni | Fe | Os |
| Standard 316 | 0.020 | 1.760 | 0.470 | 0.014 | 0.002 | 17.490 | 2.790 | 14.680 | 62.774 | 0.000 |
| Modified 316 | ≦0.030 | ≦2.000 | ≦0.750 | ≦0.023 | ≦0.010 | 12.000- | .000- | 10.000- | 46.185- | 2.000- |
| | | | | | | 20.000 | 3.000 | 18.000 | 74.000 | 10.000 |

TABLE VI

| | Component (%) | | | | | | | | | |
|---|---|---|---|---|---|---|---|---|---|---|
| | C | Mn | Si | P | S | Cr | Mo | Ni | Fe | Pd |
| Standard 316 | 0.020 | 1.760 | 0.470 | 0.014 | 0.002 | 17.490 | 2.790 | 14.680 | 62.774 | 0.000 |
| Modified 316 | ≦0.030 | ≦2.000 | ≦0.750 | ≦0.023 | ≦0.010 | 12.000- | .000- | 10.000- | 46.185- | 2.000- |
| | | | | | | 20.000 | 3.000 | 18.000 | 74.000 | 10.000 |

TABLE VII

| | Component (%) | | | | | | | | | |
|---|---|---|---|---|---|---|---|---|---|---|
| | C | Mn | Si | P | S | Cr | Mo | Ni | Fe | Pt |
| Standard 316 | 0.020 | 1.760 | 0.470 | 0.014 | 0.002 | 17.490 | 2.790 | 14.680 | 62.774 | 0.000 |
| Modified 316 | ≦0.030 | ≦2.000 | ≦0.750 | ≦0.023 | ≦0.010 | 12.000- | .000- | 10.000- | 46.185- | 2.000- |
| | | | | | | 20.000 | 3.000 | 18.000 | 74.000 | 10.000 |

TABLE VIII

| | Component (%) | | | | | | | | | |
|---|---|---|---|---|---|---|---|---|---|---|
| | C | Mn | Si | P | S | Cr | Mo | Ni | Fe | Re |
| Standard 316 | 0.020 | 1.760 | 0.470 | 0.014 | 0.002 | 17.490 | 2.790 | 14.680 | 62.774 | 0.000 |
| Modified 316 | ≦0.030 | ≦2.000 | ≦0.750 | ≦0.023 | ≦0.010 | 12.000- | .000- | 10.000- | 46.185- | 2.000- |
| | | | | | | 20.000 | 3.000 | 18.000 | 74.000 | 10.000 |

TABLE IX

| | Component (%) | | | | | | | | | |
|---|---|---|---|---|---|---|---|---|---|---|
| | C | Mn | Si | P | S | Cr | Mo | Ni | Fe | Ta |
| Standard 316 | 0.020 | 1.760 | 0.470 | 0.014 | 0.002 | 17.490 | 2.790 | 14.680 | 62.774 | 0.000 |
| Modified 316 | ≦0.030 | ≦2.000 | ≦0.750 | ≦0.023 | ≦0.010 | 12.000- | .000- | 10.000- | 46.185- | 2.000- |
| | | | | | | 20.000 | 3.000 | 18.000 | 74.000 | 10.000 |

TABLE X

| Component (%) | | | | | | | | | |
|---|---|---|---|---|---|---|---|---|---|
| C | Mn | Si | P | S | Cr | Mo | Ni | Fe | W |

| | | | | | | | | | | |
|---|---|---|---|---|---|---|---|---|---|---|
| Standard 316 | 0.020 | 1.760 | 0.470 | 0.014 | 0.002 | 17.490 | 2.790 | 14.680 | 62.774 | 0.000 |
| Modified 316 | ≦0.030 | ≦2.000 | ≦0.750 | ≦0.023 | ≦0.010 | 12.000- | .000- | 10.000- | 46.185- | 2.000- |
| | | | | | | 20.000 | 3.000 | 18.000 | 74.000 | 10.000 |

The austenitic series 300 stainless steel alloy for fabricating the present invention stent with improved radiopaque properties can contain up to 0.03% of carbon. High concentrations of the carbon element can create iron carbides which precipitate at the grain boundaries resulting in reduced mechanical and corrosion properties. Therefore, too much carbon adversely affects the fracture toughness of this alloy.

Chromium contributes to the good hardenability corrosion resistance and hardness capability of this alloy and benefits the desired low ductile-brittle transition temperature of the alloy. Therefore, at least about 12%, and preferably at least about 17.5% chromium is present. Above about 20% chromium, the alloy is susceptible to rapid overaging such that the unique combination of high tensile strength and high fracture toughness is not attainable. In addition, an alloy with a high percentage of chromium can result in the leaching of Cr ions, an element known to be toxic to human and animal tissues.

Nickel contributes to the hardenability of this alloy such that the alloy can be hardened with or without rapid quenching techniques. In this capacity, nickel benefits the fracture toughness and stress corrosion cracking resistance provided by this alloy and contributes to the desired low ductile-to-brittle transition temperature. Furthermore, nickel also is an austenitic stabilizer, thereby encouraging that during cooling process of the alloy the face-centered cubic structure is maintained. Accordingly, at least about 10.0%, and preferably at least about 14.7% nickel is present. Above about 18% nickel, the fracture toughness and impact toughness of the alloy can be adversely affected because the solubility of carbon in the alloy is reduced which may result in carbide precipitation in the grain boundaries when the alloy is cooled at a slow rate, such as when air cooled following forging. In addition, an alloy with a high percentage of nickel can result in the leaching of Ni ions, an element known to be toxic to human and animal tissues.

Therefore, using a stainless steel alloy for fabricating stents with a relatively high percent of certain components, such as nickel (Ni) or chromium (Cr) could result in leaching of Ni or Cr ions to human tissues. This leaching of toxins is exacerbated by laser cutting techniques used for fabricating stent design from tubular members. As discussed, it is well known that Ni and Cr are metallic components with toxic properties.

Molybdenum is present in this alloy because it benefits the desired low ductile brittle transition temperature of the alloy. In addition, molybdenum is a ferrite stabilizer and can have an effect on the stabilization of the desired austenitic structure. Above about 3% molybdenum the fracture toughness of the alloy is adversely affected. Preferably, molybdenum is limited to not more than about 1.2%. However, either a portion or the entire percent of the molybdenum can be replaced with certain radiopaque elements such as Pt, Au, Os, PD, or W without adversely affecting the desired characteristics of the alloy.

The austenitic 300 stainless steel alloy for fabricating the present invention stent with radiopaque properties can also contain up to 2.0% manganese. Manganese is an austenitic stabilizer and is partly depended upon to maintain the austenitic, nonmagnetic character of the alloy. Manganese encourages during cooling process of the alloy, that the face-centered cubic structure is maintained. Manganese also plays a role, in part, providing resistance to corrosive attack.

The balance of the alloy according to the present invention is essentially iron except for the usual impurities found in commercial grades of alloys intended for similar service or use. The levels of such elements must be controlled so as not to adversely affect the desired properties of this alloy. For example, phosphorus is limited to not more than about 0.008% and sulfur is limited to not more 0.004%. In addition, the alloy for fabricating a series 300 stainless steel alloy with radiopaque properties can contain up to 0.75% silicon. Furthermore, the alloy for fabricating a series 300 stainless steel stent with radiopaque properties can contain up to 0.023% and 0.002% phosphorus and sulfur, respectively, without affecting the desirable properties.

No special techniques are required in melting, casting, or working the alloy for fabricating the present invention stent. Induction heating is the preferred method of melting and refining the alloy used in fabricating the present invention stent. Arc melting followed by argon-oxygen decarburization is another method of melting and refining, but other practices can be used. In addition, this alloy can be made using powder metallurgy techniques, if desired. This alloy is also suitable for continuous casting techniques.

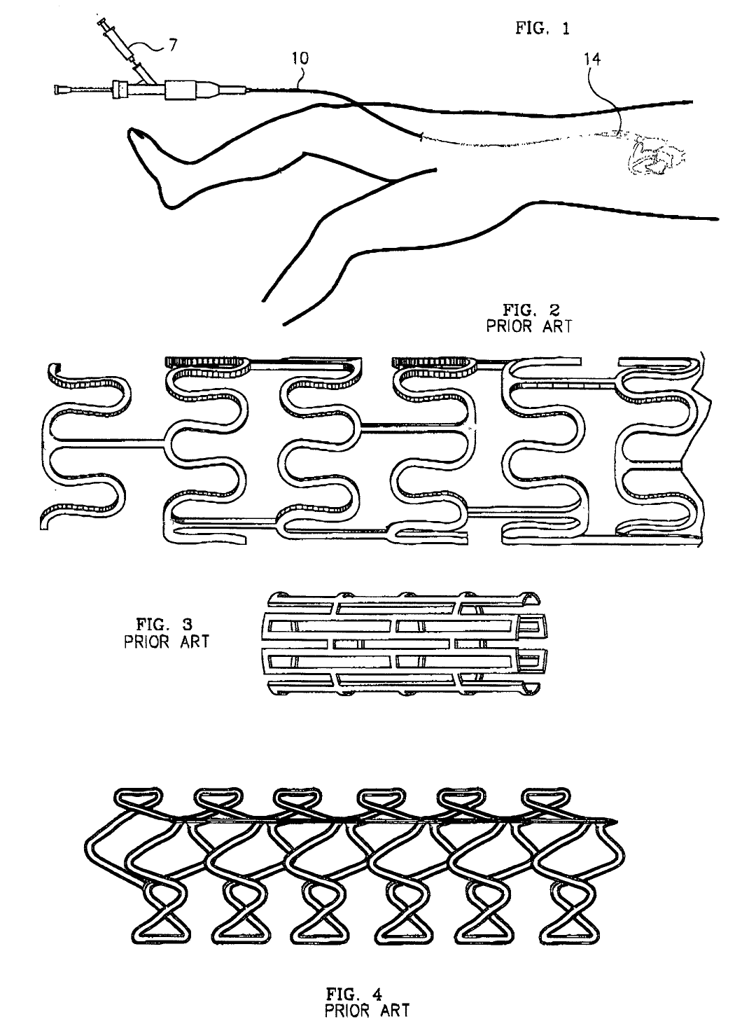

Now referring to FIG. 1, presented is an environmental example that is directed to an expandable stent which is relatively flexible along its longitudinal axis to facilitate delivery through tortuous body lumens, but which is stiff and stable enough radially in an expanded condition to maintain the patency of a body lumen such as an artery when implanted therein. As shown in FIG. 2, this invention generally includes a plurality of radially expandable loop elements which are relatively independent in their ability to expand and to flex relative to one another. Interconnecting elements or a backbone extends between the adjacent loop elements to provide increased stability and a preferable position for each loop to prevent warping of the stent upon the expansion thereof. The resulting stent structure is a series of radially expandable loop elements which are spaced longitudinally close enough so that the obstruction, vessel wall, and any small dissections located at the treatment site of a body lumen may be dilated or pressed back into position against the lumenal wall. The individual loop elements may bend relative to adjacent loop elements without significant deformation, cumulatively providing a stent which is flexible along its length and about its longitudinal axis but is still very stiff in the radial direction in order to resist collapse.

It should be noted that the stent is expanded from a contracted configuration to achieve an expanded configuration by “deforming” certain elements. By use of the term “deformed” it is meant that the material from which stent is manufactured is subjected to a force which is greater than the elastic limit of the material utilized to make expandable elements. Accordingly, the force is sufficient to permanently or semi-permanently bend the expandable elements whereby the diameter of the stent increases from the first diameter, d, to the expanded diameter, d¹. The force to be applied to the expandable elements must be sufficient to not “spring back” and assume the contracted or partially contracted configuration. Therefore, the expanded stent retains the expanded configuration and is relatively rigid in the sense of having an outer shape maintained by a fixed frame work, and not pliant.

The open reticulated structure of the stent allows for a large portion of the vascular wall to be exposed to blood which can improve the healing and repair of any damaged vessel lining. It is desirable that the stent struts be relatively thin in cross-section to minimize overall profile yet have enough radial and tensile strength to maintain vessel patency after stent deployment. In order to achieve the radiopaque characteristics desired for clinical procedures, other stent designs must compromise some of the preferred characteristics by increasing the cross-sectional thickness of all the struts, increasing the cross-sectional thickness of some of the struts, or employing other materials for fabrication that fail to have the preferred characteristics of series 300 stainless steels. By fabricating a stent design with the unique stainless steet alloy described herein, one can optimize the physical and mechanical parameters of the stent design for clinical utility without compromising the desired stent characteristics.

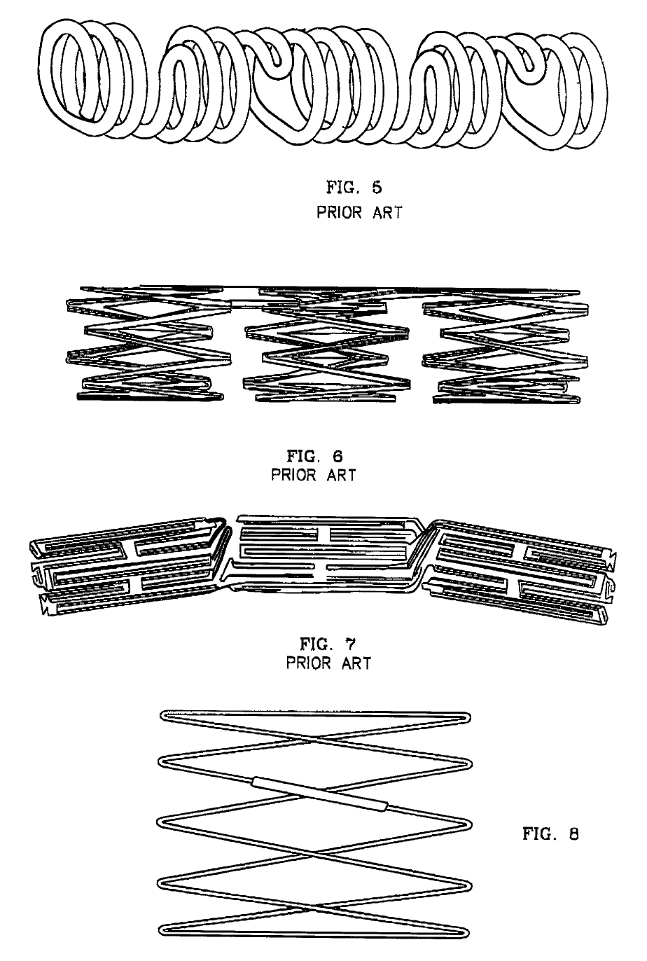

As per the stent design previously described herein, there are a variety of other stent designs. However, the present invention is not limited to any particular design. However, further examples, are shown in FIGS. 4-8, are given below to further facilitate use of the invention. U.S. Pat. Nos. 4,886,062 and 5,133,732 to Wiktor describe a stent 200 with a cylindrical body formed of generally continuous wire 210 having a deformable zigzag 220 wherein the wire is a coil of successive windings and the zigzag is in the form of a sinusoidal wave, whereby the stent body may be expanded from the first unexpended diameter to a second expanded diameter by the force of an inflating balloon. There are also means such as hooks 230 for preventing the stent body from stretching along its longitudinal axis. (See FIG. 4.) U.S. Pat. No. 4,969,458 to Wiktor shows a stent 250 which is a wire 260 winding in a hollow cylindrical shape. The winding includes a series of groups of helical coils 270 along the length of the winding while providing radial strength. The coils of each group are wound in a direction opposite to the winding of the next adjacent group of coils. A reversely turned loop 280 joining each to successive groups allows for smooth expansion of the adjacent group of coils. (See FIG. 5.) U.S. Pat. No. 5,282,823 of Schwartz shows a stent 300 comprising a cylindrical shaped body which comprises a plurality of substantially helical metal elements 310 joined to allow flexing of the stent along its longitudinal axis. The helical wire winding is substantially continuous and there is a polymeric connector 320 extending between the helical metal elements to provide strain relief means. (See FIG. 6.) U.S. Pat. No. 5,104,404 to Wolff is similar. U.S. Pat. No. 5,102,417 is similar in design to U.S. Pat. No. 5,195,984 described earlier hereinabove and assigned to the same assignee. U.S. Pat. No. 5,102,417 shows a plurality of expandable and deformable vascular grafts 330 which are thin wall tubular members 340 having a plurality of slots 350 disposed substantially parallel to the longitudinal axis of the tubular members and adjacent grafts are flexibly connected by at least one connector member 350. (See FIG. 7.) U.S. Pat. Nos. 5,102,417, 4,739,762, 4,733,665, and 4,776,337 are all by Palmaz. The Palmaz patents are similar in design to the '417 and '984 patents described earlier hereinabove. U.S. Pat. No. 4,580,568 to Gianturco describes a stent 400 comprising a wire formed into a closed zigzag configuration including an endless series of straight sections 410 and a plurality of bends 420. The straight sections are joined by the bends to form the stent. (See FIG. 8.) The stent is resiliently depressible into a small first shape wherein the straight sections are arranged side by side and closely adjacent one another for insertion into a passageway and the bends are stored stressed therein.

The present invention stent can be manufactured from a tubular member made from one of the cited stainless steel alloys described herein using a various manufacturing techniques. However, the present invention is not limited to any particular fabrication method. The following examples are given below to further facilitate use of the invention. There are several manufacturing techniques which can transform a tubular member into a particular stent design: 1) photo-mask and etch techniques as described in U.S. Pat No. 5,902,475; 2) a laser ablation/etching process disclosed in U.S. Pat. Nos. 6,066,167, 6,056,776, 5,766,238, 5,735,893, 5,514,154, or 5,421,955; and 3) utilizing a laser to directly cut away metal and form the pattern into a tubular member. Either one of these manufacturing examples can be used to produce the present invention stent with the radiopaque stainless steel alloy.

In the photo mask and etch process the outer surface of a tubular member is uniformly coated with a photo-sensitive resist. This coated tubular member is then placed in an apparatus designed to rotate the tubular member while the coated tubular member is exposed to a designated pattern of ultraviolet (UV) light. The UV light activates the photosensitive resist causing the areas where UV light is present to expose (cross-link) the photo-sensitive resist. The photo-sensitive resist forms cross links where is it exposed to the UV light thus forming a pattern of hardened and cured polymer which mimics the particular stent design surrounded by uncured polymer. The film is adaptable to virtually an unlimited number of intricate stent designs. The process from the apparatus results in the tubular member having a discrete pattern of exposed photo-sensitive material with the remaining areas having unexposed photo-sensitive resist.

The exposed tubular member is immersed in a resist developer for a specified period of time. The developer removes the relatively soft, uncured photo-sensitive resist polymer and leaves behind the cured photo-sensitive resist which mimics the stent pattern. Thereafter, excess developer is removed from the tubular member by rinsing with an appropriate solvent. At this time, the entire tubular member is incubated for a specified period of time, allowing the remaining photo-sensitive resist polymer to fully cure and bond to the surface of the processed tubular member.

The processed tubular member is then exposed to an electrochemical etching process which removes uncovered metal from the tubular member, resulting in the final tubular member or stent configuration.

In an example of the laser/etching process, a tubular member is coated with a resist and placed in a rotatable collet fixture of a machine controlled apparatus for positioning the tubular member relative to a laser. Then, according to the machine coded instructions, the tubing is rotated and moved longitudinally relative to the laser which is also machine controlled whereby the laser selectively removes the resistant coating on the tubular member by ablation. A stent pattern is formed on the surface of the tubular member that is created by a subsequent chemical etching process.

In an example of the direct laser method, a tubular member is placed in a collet fixture of a machine controlled apparatus for positioning the tubular member relative to a laser. Then, according to the machine coded instructions, the tubing is rotated and moved longitudinally relative to the laser which is also machine controlled whereby the laser selectively ablates and removes metal forming the stent pattern.

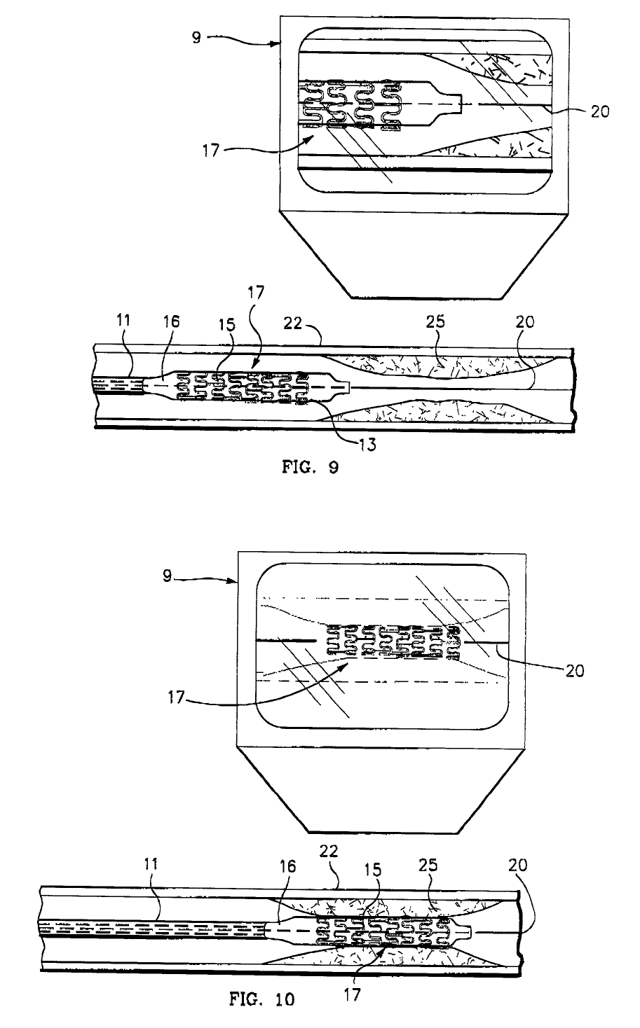

FIG. 9 presents a schematic view of the present invention showing the radiopaque stent 17 in a proximal position to the lesion 25. Attached to distal catheter shaft 11 is the delivery balloon with proximal end 16 and distal end 13. Mounted on the delivery balloon is a representation of the present invention stent 17 with unexpended struts 15 in a non-expanded configuration. A previously placed guidewire 20 transects the catheter shaft and extends beyond lesion 25. Also shown is the corresponding image of the proximally placed radiopaque stent 17 on a typical cine angiogram or fluoroscopic equipment 9.

In the next stent of a typical clinical procedure, the stent/delivery system is advanced to a position where it becomes centered within the lesion 25 (FIG. 10). Also shown is the corresponding image of the contracted radiopaque stent centered within the lesion on a typical cine angiogram or fluoroscopic equipment.

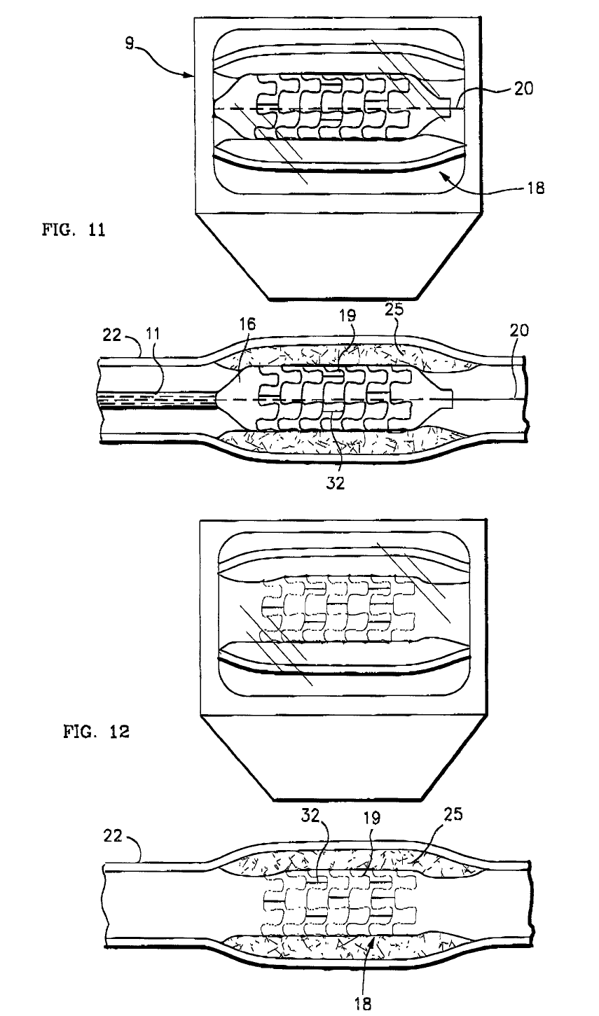

FIG. 11 is a schematic view of the present invention showing the radiopaque stent and delivery catheter centered within the lesion in an expanded configuration. Also shown is the corresponding image of the expanded radiopaque stent centered within the lesion on a typical cine angiogram or fluoroscopic equipment.

FIG. 12 is a schematic view of the present invention showing the expanded and deployed radiopaque stent embedded within the lesion. Also shown is the corresponding image of the embedded radiopaque stent deployed within the lesion on a typical cine angiogram or fluoroscopic equipment.

It is apparent from the foregoing description and the accompanying examples, that the alloy according to the present invention provides a unique combination of tensile strength and radiopaque characteristics not provided by known series 300 stainless steel alloys. This stent invention is well suited to applications where high strength, biocompatibility, and radiopacity are required.

The terms and expressions which have been employed herein are used as terms of description and not of limitation. There is no intention in the use of such terms and expressions to exclude any equivalents of the features described or any portions thereof. It is recognized, however, that various modifications are possible within the scope of the invention claimed.

While the invention has been illustrated and described herein in terms of its use as an intravascular stent, it will be apparent to those skilled in the art that the stent can be used in other instances such as to expand prostate urethras in cases of prostate hyperplasia. Other modifications and improvements may be made without departing from the scope of the invention.

Other modifications and improvements can be made to the invention without departing from the scope thereof.

We claim:

1. An intravascular stent manufactured from a stainless steel alloy which provides increased radiopaque characteristics, said alloy consisting essentially of, in weight percent, about

| C | Mn | Si | P | S | Cr | Mo | Ni | Au |
|---|---|---|---|---|---|---|---|---|
| ≦0.030 | ≦2.000 | ≦0.750 | ≦0.023 | ≦0.010 | 12.000- | 2.000- | 10.000- | 2.000- |
| | | | | | 20.000 | 3.000 | 18.000 | 10.000 |

and the balance is essentially iron.

2. An intravascular stent manufactured from a stainless steel alloy which provides increased radiopaque characteristics, said alloy consisting essentially of, in weight percent, about

| C | Mn | Si | P | S | Cr | Mo | Ni | Os |
|---|---|---|---|---|---|---|---|---|
| ≦0.030 | ≦2.000 | ≦0.750 | ≦0.023 | ≦0.010 | 12.000- | 2.000- | 10.000- | 2.000- |
| | | | | | 20.000 | 3.000 | 18.000 | 10.000 |

and the balance is essentially iron.

3. An intravascular stent manufactured from a stainless steel alloy which provides increased radiopaque characteristics, said alloy consisting essentially of, in weight percent, about

| C | Mn | Si | P | S | Cr | Mo | Ni | Pd |
|---|---|---|---|---|---|---|---|---|
| ≦0.030 | ≦2.000 | ≦0.750 | ≦0.023 | ≦0.010 | 12.000- | 2.000- | 10.000- | 2.000- |
| | | | | | 20.000 | 3.000 | 18.000 | 10.000 |

and the balance is essentially iron.

4. An intravascular stent manufactured from a stainless steel alloy which provides increased radiopaque characteristics, said alloy consisting essentially of, in weight percent, about

| C | Mn | Si | P | S | Cr | Mo | Ni | Pt |
|---|---|---|---|---|---|---|---|---|
| ≦0.030 | ≦2.000 | ≦0.750 | ≦0.023 | ≦0.010 | 12.000- | 2.000- | 10.000- | 2.000- |
| | | | | | 20.000 | 3.000 | 18.000 | 10.000 |

and the balance is essentially iron.

5. An intravascular stent manufactured from a stainless steel alloy which provides increased radiopaque characteristics, said alloy consisting essentially of, in weight percent, about

| C | Mn | Si | P | S | Cr | Mo | Ni | Re |
|---|---|---|---|---|---|---|---|---|
| ≦0.030 | ≦2.000 | ≦0.750 | ≦0.023 | ≦0.010 | 12.000- | 2.000- | 10.000- | 2.000- |
| | | | | | 20.000 | 3.000 | 18.000 | 10.000 |

and the balance is essentially iron.

6. An intravascular stent manufactured from a stainless steel alloy which provides increased radiopaque characteristics, said alloy consisting essentially of, in weight percent, about

| C | Mn | Si | P | S | Cr | Mo | Ni | Ta |
|---|---|---|---|---|---|---|---|---|
| ≦0.030 | ≦2.000 | ≦0.750 | ≦0.023 | ≦0.010 | 12.000- | 2.000- | 10.000- | 2.000- |
| | | | | | 20.000 | 3.000 | 18.000 | 10.000 |

and the balance is essentially iron.

7. An intravascular stent manufactured from a stainless steel alloy which provides increased radiopaque characteristics, said alloy consisting essentially of, in weight percent, about

| C | Mn | Si | P | S | Cr | Mo | Ni | W |
|---|---|---|---|---|---|---|---|---|
| ≦0.030 | ≦2.000 | ≦0.750 | ≦0.023 | ≦0.010 | 12.000- | 2.000- | 10.000- | 2.000- |
| | | | | | 20.000 | 3.000 | 18.000 | 10.000 |

and the balance is essentially iron.

8. An intravascular stent manufactured from a stainless steel alloy which provides increased radiopaque characteristics, said alloy consisting essentially of, in weight percent, about

| C | Mn | Si | P | S | Cr | Mo | Ni | Fe | “X” |
|---|---|---|---|---|---|---|---|---|---|
| ≦0.030 | ≦2.000 | ≦0.750 | ≦0.023 | ≦0.010 | 12.000- | 2.000- | 10.000- | 46.185- | 2.000- |
| | | | | | 20.000 | 3.000 | 18.000 | 10.000 | |

whereby variable “X” could be comprised from a group consisting of Au, Os, Pd, Pt, Re, Ta, or W.

9. An intravascular stent manufactured from a stainless steel alloy which provides increased radiopaque characteristics, said alloy consisting essentially of, in weight percent, about

| C | Mn | Si | P | S | Cr | Mo | Ni | Fe | “X” |
|---|---|---|---|---|---|---|---|---|---|
| ≦0.030 | ≦2.000 | ≦0.750 | ≦0.023 | ≦0.010 | 12.000- | 2.000- | 10.000- | 46.185- | 2.000- |
| | | | | | 20.000 | 3.000 | 18.000 | 10.000 | |

whereby variable “X” could be comprised from a group consisting of Gold, Osmium, Palladium, Platinum, Rhenium, Tantalum, or Tungsten.

10. An intravascular stent fabricated for a modified a stainless steel alloy which provides increased radiopaque characteristics over standard 300 series stainless steel.

11. An intravascular stent as recited in claim 9, wherein a portion of Gold replaces a portion of Iron.

12. An intravascular stent as recited in claim 9, wherein a portion of Gold replaces a portion of Molybdenum.

13. An intravascular stent as recited in claim 9, wherein a portion of Gold replaces a portion of both Iron and Molybdenum.

14. An intravascular stent as recited in claim 9, wherein a portion of Osmium replaces a portion of Iron.

15. An intravascular stent as recited in claim 9, wherein a portion of Osmium replaces a portion of Molybdenum.

16. An intravascular stent as recited in claim 9, wherein a portion of Osmium replaces a portion of both Iron and Molybdenum.

17. An intravascular stent as recited in claim 9, wherein a portion of Palladium replaces a portion of Iron.

18. An intravascular stent as recited in claim 9, wherein a portion of Palladium replaces a portion of Molybdenum.

19. An intravascular stent as recited in claim 9, wherein a portion of Palladium replaces a portion of both Iron and Molybdenum.

20. An intravascular stent as recited in claim 9, wherein a portion of Platinum replaces a portion of Iron.

21. An intravascular stent as recited in claim 9, wherein a portion of Platinum replaces a portion of Molybdenum.

22. An intravascular stent as recited in claim 9, wherein a portion of Platinum replaces a portion of both Iron and Molybdenum.

23. An intravascular stent as recited in claim 9, wherein a portion of Rhenium replaces a portion of Iron.

24. An intravascular stent as recited in claim 9, wherein a portion of Rhenium replaces a portion of Molybdenum.

25. An intravascular stent as recited in claim 9, wherein a portion of Rhenium replaces a portion of both Iron and Molybdenum.

26. An intravascular stent as recited in claim 9, wherein a portion of Tantalum replaces a portion of Iron.

27. An intravascular stent as recited in claim 9, wherein a portion of Tantalum replaces a portion of Molybdenum.

28. An intravascular stent as recited in claim 9, wherein a portion of Tantalum replaces a portion of both Iron and Molybdenum.

29. An intravascular stent as recited in claim 9, wherein a portion of Tungsten replaces a portion of Iron.

30. An intravascular stent as recited in claim 9, wherein a portion of Tungsten replaces a portion of Molybdenum.

31. An intravascular stent as recited in claim 9, wherein a portion of Tungsten replaces a portion of both Iron and Molybdenum.
